(19) United States
(12) Patent Application Publication
(10) Pub. No.: US 2018/0289354 A1
(43) Pub. Date: Oct. 11, 2018

(54) ULTRASOUND APPARATUS AND METHOD FOR DETERMINING A MEDICAL CONDITION OF A SUBJECT

(71) Applicant: KONINKLIJKE PHILIPS N.V., EINDHOVEN (NL)

(72) Inventors: Nemanja CVIJANOVIC, EINDHOVEN (NL); Patrick KECHICHIAN, EINDHOVEN (NL)

(21) Appl. No.: 15/763,505

(22) PCT Filed: Sep. 30, 2016
(86) PCT No.: PCT/EP2016/073425

§ 371 Date: Mar. 27, 2018

(30) Foreign Application Priority Data
Sep. 30, 2015 (EP) 15187617.4

(51) Int. Cl. A61B 8/08

(52) U.S. Cl. CPC A61B 8/08; A61B 5/4082; A61B 8/488

(57) ABSTRACT

The present invention relates to an ultrasound apparatus (10) for determining a medical condition of a subject (12), comprising: an ultrasound acquisition unit (14) for receiving ultrasound waves (23) from the subject and for providing an ultrasound signal (24) corresponding to articulatory movements of the subject. A processing unit (26) is coupled to the ultrasound acquisition unit for determining a frequency change of the ultrasound signal. An evaluation module (32) evaluates the ultrasound signal and determines the medical condition of the subject on the basis of the frequency change of the ultrasound signal.

### FIELD OF THE INVENTION

The present invention relates to an ultrasound apparatus for determining a medical condition of a subject on the basis of articulatory movements. The present invention further relates to a method for determining a medical condition of a subject on the basis of articulatory movements. The present invention further relates to an ultrasound system for determining a medical condition of a subject and to a computer program for carrying out method steps to determine a medical condition of a subject.

### BACKGROUND OF THE INVENTION

Numerous degenerative health disorders such as neurological disease which affects the motoric functions of a person, e.g. due to Parkinson's disease, result in speech impairment of the patient. The progression of the impairment can increase over time and a monitoring of the progression of the disease can be important for the diagnosis or for evaluating the effectiveness of a physical or a pharmaceutical therapy.

Audio speech analysis for monitoring diseases like Parkinson's disease based on acoustic information and using physiological techniques is well known in the art, however, this technique requires a high level of cooperation of the patient which might not always be possible and is uncomfortable for the user. Further, an objective measurement result which may be used precisely to determine a progression of the disease cannot be achieved. Other techniques are based on electromagnetic articulography and utilize strain gauges which are intrusive due to the required skin contact and uncomfortable for the user.

It is further known to utilize features extracted from ultrasound reflection signals for automatic speech and speaker recognition, and voice activity detection.

From McLoughlin et al.: “Mouth state detection from low frequency ultrasound reflection”, circuits, systems and signal processing, Cambridge, Miss., US, volume 34, No. 4, 9 Oct. 2014, pages 1279-1304 discloses a non-contact low frequency ultrasound method, which can determine from airborne reflection whether the lips of a subject are open or closed. The method is capable of accurately distinguishing between open and closed lip states through the use of a low-complexity detection algorithm, and is highly robust to interfering audible noise.

### SUMMARY OF THE INVENTION

It is an object of the present invention to provide an ultrasound apparatus which can detect articulatory movements precisely and comfortably for the user in order to determine a medical condition of a subject. It is a further object of the present invention to provide a corresponding ultrasound system for determining a medical condition of a subject. It is a further object of the present invention to provide a corresponding method for determining a medical condition of a subject and a computer program for implementing such method.

In a first aspect of the present invention an ultrasound apparatus for determining a medical condition of a subject is provided, comprising:

an ultrasound acquisition unit for receiving ultrasound waves reflected from the subject and for providing an ultrasound signal corresponding to articulatory movements of the subject,

a processing unit coupled to the ultrasound acquisition unit for determining a frequency change of the ultrasound signal over time, and

an evaluation module for evaluating the ultrasound signal over time, preferably the frequency change of the ultrasound signal over time, and for determining the medical condition of the subject on the basis of the frequency change of the ultrasound signal over time.

In a further aspect of the present invention a method for determining a medical condition of a subject is provided comprising the steps of:

receiving an ultrasound signal from an ultrasound acquisition unit corresponding to articulatory movements of a subject,

determining a frequency change of the ultrasound signal, and

evaluating the ultrasound signal and determining the medical condition of the subject on the basis of the frequency change of the ultrasound signal.

In a further aspect of the present invention an ultrasound system for determining a medical condition of a subject is provided, comprising:

an ultrasound transducer including an ultrasound emitter for emitting ultrasound waves and an ultrasound acquisition unit for receiving ultrasound waves reflected from the subject and for providing an ultrasound signal corresponding to articulatory movements of the subject,

a processing unit coupled to the ultrasound acquisition unit for determining a frequency change of the ultrasound signal over time,

an evaluation module connected to the processing unit for determining the medical condition of the subject,

a data interface for connecting the evaluation unit to a storage unit and for receiving results of a previous measurement of articulatory movements of the subject, wherein the evaluation module is adapted to determine the medical condition of the subject on the basis of the frequency change of the ultrasound signal over time and the results of the previous measurement.

In a further aspect of the present invention, there is provided a computer program product evaluating the frequency change of the ultrasound signal and for determining the medical condition of the subject on the basis of the frequency change of the ultrasound signal over time, comprising a computer readable medium having computer readable code embodied therein, the computer readable code being configured such that, on execution by a suitable computer or processing unit, the computer or processing unit is caused to perform any of methods described above. The computer program product can comprise computer-readable program code downloadable from or downloaded from a communications network, or storable on, or stored on a computer-readable storage medium, which computer-readable program code, when run on a computer or processing unit causes the computer or processing unit to perform the steps of any embodiments of the method in accordance with the present invention. The computer program product can be suitable to work with a system including a server device and a client device. Part of the steps can be performed on the server device while the other or another part of the steps is performed on the client device. The server and client device can be remote from each other and connected through wired or wireless communication as known in the art. Alternatively, all steps are performed on a server device or on a client device. The server device and client device can have communication devices for communicating with each other using wired or wireless communication protocols.

Preferred embodiments of the invention are defined in the dependent claims. It shall be understood that the claimed method has similar and/or identical preferred embodiments as the claimed device and as defined in the dependent claims.

The present invention is based on the idea that movements of the face of the subject are determined on the basis of ultrasound waves. The movements of the face in the direction of the ultrasound waves cause a shift in the frequency of the backscattered ultrasound waves due to the Doppler Effect so that movements parallel to the incident ultrasound beams can be detected. The so detected movements of the face correspond to the articulatory movements of the subject so that an objective and reproducible evaluation of the articulatory movements can be provided. Further, since the detection is ultrasound-based and the movements in the direction of the ultrasound waves are detected, the subject or patient to be measured can speak normally and no sustained phonation is required. Hence, the comfort of the measurement is substantially improved. Hence, a precise and comfortable measurement of the articulatory movements can be provided for monitoring of a neurological disease. As a consequence of the measurement of the change of frequency of the ultrasound signal over time, a change or a progression of neurological condition or disease, such as, for instance, Parkinson, Multiple Sclerosis (MS), Motor Neurone Disease (Amyotrophic Lateral Sclerosis), Epilepsy, or a plurality of other conditions or diseases can be detected.

In an embodiment of the invention the frequency change of the ultrasound signal corresponds to a movement of the subject or the movement of a portion of the subject with respect to the ultrasound acquisition unit. This is a possibility to determine the articulatory movement of the subject precisely with low technical effort.

In an embodiment of the present invention, the ultrasound acquisition unit is adapted to receive the ultrasound waves without requiring contact to the subject. In other words, the ultrasound acquisition unit is adapted to receive the ultrasound waves contactless or contact free. The ultrasound acquisition unit is preferably aimed at the subject's mouth in order to capture the articulatory movement, wherein the distance between the ultrasound acquisition unit and the subject is preferably between 20 and 100 cm. This is a possibility to provide a non-intrusive measurement which is comfortable for the user, since the user does not need to wear sensors or needs to be contacted to measure units.

In an embodiment of the present invention, the processing unit is adapted to determine an amplitude and/or a velocity of the articulatory movement on the basis of the ultrasound signal and to determine the medical condition on the basis of the amplitude and/or the velocity. This is a possibility to extract a certain parameter from the ultrasound signal with low technical effort so that the results of the measurements are reproducible and can be objectively compared with other measurements.

In an embodiment of the present invention, the processing unit comprises a frequency analysis unit for determining different frequency values of the ultrasound signal and for determining the medical condition on the basis of the different frequency values. This is a possibility to evaluate the ultrasound signal in the frequency domain effectively with low technical effort, e.g., on the basis of a Fourier transformation.

In an embodiment of the present invention, the processing unit is further adapted to determine a cumulative energy of the ultrasound signal and to determine the medical condition on the basis of the cumulative energy. This is a possibility to determine a single parameter which is indicative of the Doppler shifts resulting from movements of the subject, so that changes in frequency information and energy can be considered to determine the medical condition.

It is further preferred if the processing unit is adapted to determine the cumulative energy of the ultrasound signal as a time-dependent cumulative frequency energy. This is a possibility to determine a change of the speech energy in the respective frequency bands over time so that the preciseness of the measurement can be improved.

In a preferred embodiment, the processing unit is adapted to determine an energy spectrum on the basis of the ultrasound signals and to determine the medical condition on the basis of the energy spectrum. This is a possibility to provide a robust detection of articulatory degradation and to provide a robust monitoring of a disease of the subject.

In a further preferred embodiment, the processing unit is adapted to determine a variance value of the energy spectrum and to determine the medical condition on the basis of the variance value. This is a possibility to determine a further objective parameter from the energy spectrum of the ultrasound signal so that a reproducible comparison to other measurements can be provided.

In an embodiment, the ultrasound apparatus further comprises a data interface for connecting the evaluation unit to a storage unit and for receiving results of previous measurements of articulatory movements of the subject, wherein the evaluation unit is adapted to determine the medical condition of the subject on the basis of the frequency change and the results of the previous measurement. This is a possibility to determine a progress of a disease or an effectiveness of a therapy with low technical effort.

In an embodiment of the invention, the ultrasound apparatus further comprises a sound detection unit for detecting sound received from the subject and for determining an acoustic activity of the subject. This is a possibility to determine a speech activity of the subject and to exclude that other movement activities of the subject are detected. This is a possibility to avoid erroneous measurements. Furthermore, it is possible to include non-articulatory movement detectors based on the received ultrasound signal, to ensure that only articulatory movements are monitored.

In an embodiment, the evaluation unit comprises an evaluation model for determining the medical condition of the subject on the basis of the frequency change of the ultrasound signal. This is a possibility to utilize certain classifications and/or experiences or indication to determine an impairment of the articulatory movement and to determine whether a positive or negative progression of the disease is present. This is a possibility to improve the evaluation of the measurements in general. In an embodiment, the evaluation model comprises an equation or a characteristic curve including the medical condition or a speech impairment as a function of the frequency change or a characteristic parameter derived from the frequency change.

As mentioned above, the ultrasound apparatus can determine movements of the face of the subject to be measured based on the evaluation of the ultrasound signal and on the basis of the determined frequency change of the ultrasound signal so that the articulatory movements of the subject can be precisely and reproducibly determined. Since the measurement is based on ultrasound waves received from the subject, the measurement can be performed comfortably for the user without the use of contact sensors or the like so that a non-intrusive measurement can be achieved. The evaluation on the basis of the frequency domain of the ultrasound signal in particular provides the possibility to determine the movement of the subject parallel to the propagation direction of the incident ultrasound waves so that a precise measurement and a robust determination of the medical condition of the subject can be achieved.

### BRIEF DESCRIPTION OF THE DRAWINGS

These and other aspects of the invention will be apparent from and elucidated with reference to the embodiment(s) described hereinafter. In the following drawings

FIG. 1 shows a schematic representation of an ultrasound apparatus for determining medical conditions of a subject on the basis of articulatory movements;

FIG. 2 shows a detailed schematic diagram of the ultrasound apparatus including a frequency analysis unit;

FIGS. 3A, B, show two spectrograms of an ultrasound signal of different medical conditions of the subject;

FIGS. 4A, B show different speech energy diagrams of different medical conditions of the subject; and

FIG. 5 shows different speech energy spectrums of different medical conditions of the subject.

### DETAILED DESCRIPTION OF THE INVENTION

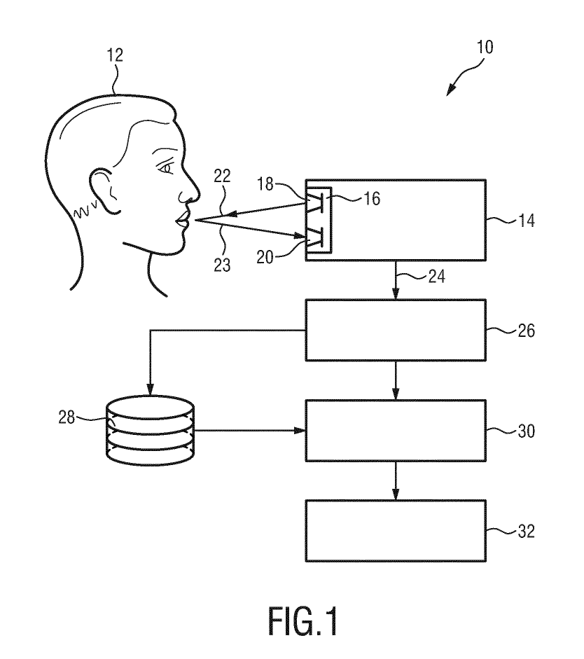

FIG. 1 shows a schematic diagram of an ultrasound apparatus generally denoted by 10. The ultrasound apparatus 10 is applied to inspect a face of a subject 12 or a patient 12. The ultrasound apparatus 10 comprises an ultrasound unit 14 having at least one ultrasound transducer 16 including one or more transducer elements 18, 20 for transmitting and receiving ultrasound waves 22.

The ultrasound transducer 16 is directed to the face or the mouth of the subject 12, wherein one of the transducer elements 18 emits the ultrasound waves 22 to the face of the subject 12 and one of the transducer elements 20 receives the ultrasound waves 23 reflected or backscattered from the face of the subject 12. The movement of the face during articulation of the subject 12, i.e. during speech production, causes a shift in the frequency of the reflected or backscattered ultrasound waves 23 due to the Doppler Effect so that the ultrasound waves received by the transducer element 20 include information about the articulatory movements of the subject 12 which can be measured by the ultrasound unit 14. Over time, changes in the velocity and the amplitude of the articulatory movements can be detected and extracted from the reflected or backscattered ultrasound waves 23 as described in the following.

On the basis of the received backscattered or reflected ultrasound waves 23, the ultrasound unit 14 provides an ultrasound signal 24 corresponding to or including the articulatory movements of the subject 12 in general.

In a preferred embodiment, the transducer 16 comprises a pair of a single ultrasound emitter and single ultrasound receiver element as shown in FIG. 1.

The backscattered or reflected ultrasound waves 23 differ from the emitted ultrasound waves 22 due to the Doppler Effect, wherein a change in the frequency of the ultrasound waves 22 result from the motion of the subject 12 or a portion of the subject 12, e.g. the mouth or the lips. The altered frequency fᵣ can be calculated by:

$${f}_{r} = \frac{c + v}{c - v} \approx ( 1 + \frac{2 v}{c} ) {f}_{c} = {f}_{c} + Δ f$$

wherein f_{c} is the frequency of the emitted ultrasound waves 22, v is the velocity of the subject 12 or a portion of the subject 12 to be measured, c the speed of the sound and Δf the Doppler shift resulting from the movement of the subject 12. The velocity v is assumed to be positive for a movement towards the transducer 16 and negative away from the ultrasound transducer 16. Due to the physics of the Doppler Effect only movement component parallel to the propagation direction of the ultrasound waves 22 can result in a Doppler shift.

In certain embodiments, the ultrasound acquisition unit 14 may also include a temperature sensor to estimate the value of the speed of the sound c to produce a reliable estimate of the Doppler shifts fᵣ.

The ultrasound acquisition unit 14 is steered or directed to the face or the mouth of the subject 12 in order to capture the articulatory movement of the subject 12. The steering can be implemented mechanically, or in software. A distance of the transducer 16 to the subject 12 can be between 20 and 100 cm depending on the level of the emitted ultrasound waves 22 and the beam width of the emitting transducer element 18. A distance between the subject 12 and the transducer 16 is preferably 50 cm. The ultrasound acquisition unit 14 can be mounted on a computer monitor or a separate stand in order to measure the articulatory movements of the subject 12 and to provide the ultrasound signal 24.

In a further preferred embodiment of the current invention, the distance between the user and the ultrasound apparatus 14 is kept relatively constant between measurement sessions for accurate comparison and/or tracking of the medical condition. For this purpose a chin rest can be used. Furthermore, the distance between the ultrasound unit 14 and the subject 12 can be calculated before the measurement begins by operating the ultrasound transmitter 18 in pulsed mode and calculating the travel time of the ultrasound pulse between user and device as received by the ultrasound receiver 20. This distance can be presented on a monitor so the user can comfortably adjust their position before activating the monitoring system.

The ultrasound apparatus 10 further comprises a processing unit 26 which is coupled to the ultrasound acquisition unit 14 and receives the ultrasound signal 24 from the ultrasound acquisition unit 14. The processing unit determines the frequency change of the ultrasound signal based on the Doppler shift and determines at least one characteristic parameter of the frequency change which is characteristic for the articulatory movement and can be compared to other measurements or utilized for determining the medical condition of the subject 12.

The characteristic parameter which is determined from the ultrasound signal 24 may be the signal energy in the frequency band beside the carrier frequency of the emitted ultrasound waves 22. In an embodiment of the invention, the characteristic parameter is determined as an average cumulative energy in the frequency band outside the carrier frequency of the emitted ultrasound waves 22 over time. A drop or a reduction of this average cumulative energy can be observed for an impaired articulation as described in the following. In another embodiment an average energy spectrum over all frequency bands is extracted as a characteristic parameter from the ultrasound signal 24.

In a further embodiment a variance value of the energy spectrum is determined as the characteristic parameter to determine the medical condition of the subject 12.

The result of the determination of the frequency change of the ultrasound signal and the extracted characteristic parameter may be stored in a database 28 for further evaluation. The results and the characteristic parameter may be stored including the time of the measurement including the day, the month and the year or an identification number to identify the respective measurement. Furthermore, an audio signal recorded by a microphone can also be stored for possible future analysis, such as comparing acoustic energy from a microphone to articulatory energy from the analyzed ultrasound signal.

It can be appreciated by those knowledgeable in the prior art that the database 28 may be stored physically in the ultrasound apparatus 10 or may be stored in a different location like a remote cloud database server.

The ultrasound apparatus 10 may comprise a comparator module 30, which is connected to the processing unit 26 or may be a part of the processing unit 26 and receives the characteristic parameter determined on the basis of the frequency change of the ultrasound signal 24. The comparator module 30 is connected to the database 28 and compares the characteristic parameter received from the processing unit 26 and a characteristic parameter from the database 28 which has been determined during a previous measurement of the articulatory movements of the subject 12. The comparator module 30 compares the characteristic parameter from the processing unit 26 and the database 28 and determines a corresponding comparator value. The ultrasound apparatus 10 further comprises an evaluation module 32 which is connected to the processing unit 26 and to the comparator module 30 or which may be a part of the processing unit 26.

In a further preferred embodiment, the energy of the reflected ultrasound carrier signal when the subject is not moving is used to normalize the features calculated by the processing unit 26 so that comparisons of the resulting features with those calculated in earlier sessions and stored in the database 28 can be accurately compared.

The evaluation module 32 evaluates the characteristic parameter received from the processing unit 26 and determines the medical condition of the subject 12 on the basis of the ultrasound signal 26. The evaluation module 32 determines whether the articulatory movements are unimpaired or impaired articulation. For the case that the ultrasound apparatus 10 comprises the comparison module 30 which provides the comparator value, the evaluation module determines whether the articulatory movements show an impairment which is degraded or whether the impairment has improved e.g. due to the administration of drugs or physiotherapy.

The evaluation module 32 may incorporate models of speech impairment for certain conditions related to the extracted characteristic features to determine whether positive or negative progression of the impairment is occurring. These models may be based on an additional database and a classification performed by experts which may be continuously updated. The models may also be stored in a cloud or an external database.

In an embodiment, the models of speech impairment are based on feature data tagged by experts based on impairment severity and/or medical condition. These data clusters may then be used to classify the medical condition of a patient based on the patient's extracted features. Methods that can be used for classification include k-nearest neighbours (KNN).

In another embodiment, feature data related to speech impairment is collected over a large population of patients afflicted with a particular medical condition, e.g., Parkinson's disease, and the severity of speech impairment assessed by one or more experts. A model is then estimated based on a best fit curve of these points on a feature vs. severity plot, e.g. linear regression or higher order regression. In other words, an equation is estimated to model the severity of speech impairment dependent on the extracted characteristic features.

The evaluation module 32 may be connected to a monitor (not shown) for displaying the results of the evaluation and the results of the measurements of the articulatory movements.

In an embodiment, the ultrasound apparatus 10 further comprises a microphone for measuring acoustic signals received from the subject 12. The microphone is connected to the processing unit 26. The processing unit 26 determines whether a voice activity or a speech activity of the subject can be detected in order to determine whether the subject 12 is actually speaking or whether the subject is somehow otherwise moving. This can exclude erroneous measurements of articulatory movements. The microphone preferably captures signals in a large bandwidth, e.g. in the hearing range of human beings between 20 and 20000 Hz.

In an embodiment, the processing unit 26 also includes algorithms to analyze the ultrasound signal and determine if significant non-articulatory movements are made in which case the processing unit 26 can either compensate for these or prompt the subject 12 to repeat the measurement.

In an embodiment where the ultrasound apparatus 10 further comprises a microphone for measuring acoustic signals received from the subject 12, the evaluation module may store models that include both acoustic and articulatory domain features to determine a given medical condition and/or the progression of the subject's speech impairment. An example of such a feature may include, but is not limited to the ratio of acoustic to articulatory energy for example.

The ultrasound apparatus 10 may further comprise a display for presenting a sequence of words or expressions to the subject 12 so that the subject 12 can repeat and articulate the respective words displayed by the display unit. This allows the system to improve the reproducibility of the measurements, since it simplifies the comparison between extracted characteristic features over time. Furthermore, headphones or loudspeakers can be used where a pre-recorded audio signal prompts the subject 12 to repeat the words or phrases in the test sequence.

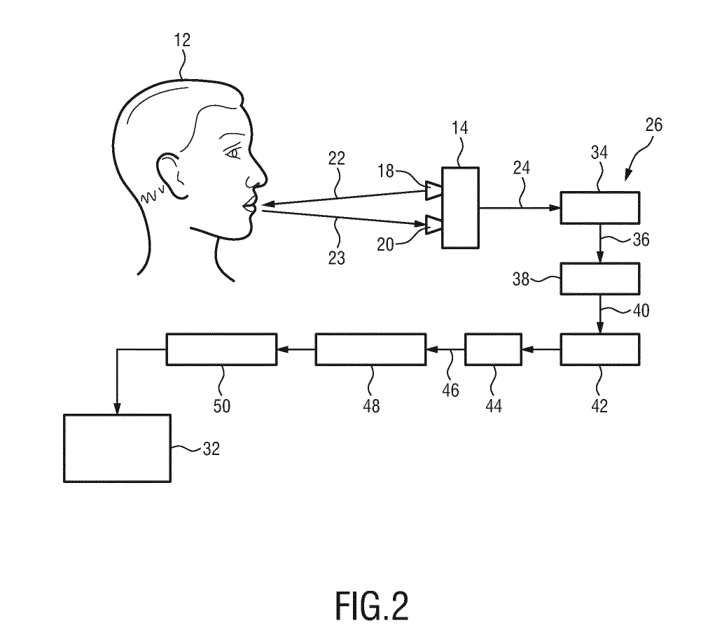

FIG. 2 shows a detailed schematic block diagram of the processing unit 26, which is connected to the ultrasound acquisition unit 14.

The processing unit 26 receives the ultrasound signal 24 from the ultrasound acquisition unit 14 corresponding to the articulatory movements of the subject 12 as described above. The processing unit 26 comprises a mixing module 34 for downmixing the ultrasound signal 24 from e.g. a carrier frequency of 40 kHz to a downmixed frequency of 4 kHz. The downmixed signal 36 is provided to a resample module 38 which resamples the downmixed signal 36 to avoid aliasing. The resampled signal 40 is provided to a segmentation module 42 for determining time blocks of the respective time-dependent resampled signal 40 and provides the time blocks to a frequency analysis unit 44. The frequency analysis unit 44 performs a Fourier transformation and in particular a Fast Fourier Transformation (FFT) and provides the frequency blocks 46 to a block analysis unit 48 which determines frequency energy of the frequency blocks 46. An extraction module 50 extracts the characteristic parameter from the frequency energy and provides the characteristic parameter to the evaluation module 32.

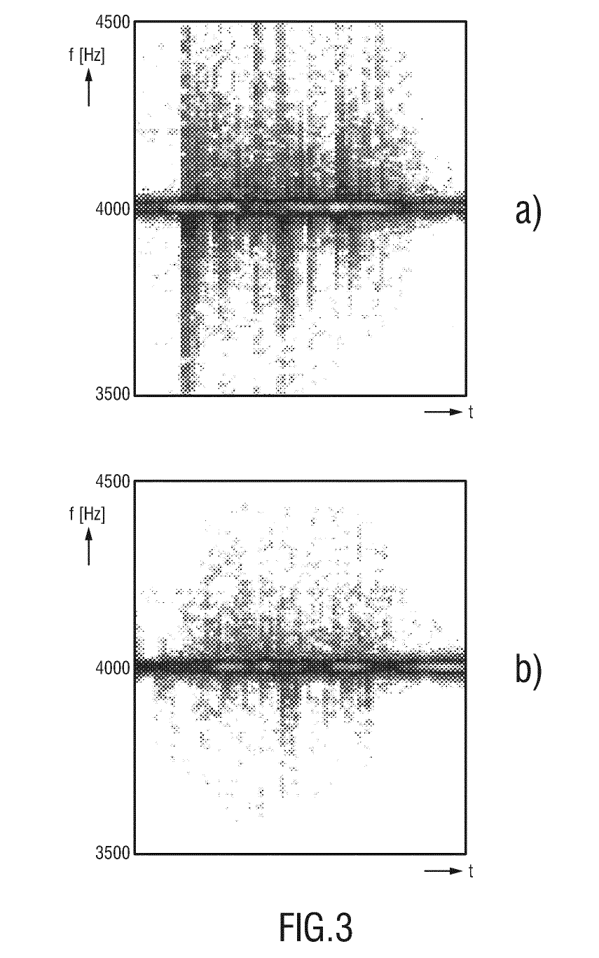

FIGS. 3A, B show a spectrogram of the ultrasound signal 24. The frequency of the ultrasound signal 24 is shown time-dependent, wherein FIG. 3A shows the ultrasound signal 24 of a normal (unimpaired) articulatory movement and FIG. 3B shows the ultrasound signal 24 of an impaired articulatory movement of the subject 12. It can be seen from FIGS. 3A and B that the impaired articulatory movement leads to a decreased velocity and amplitude of the detected movement, which results in a smaller Doppler shift and a reduced frequency energy beside the carrier band of the ultrasound waves 23. For the evaluation of the ultrasound signal 24, the frequency energy in the frequency bands beside the carrier band (4 kHz) of the ultrasound waves 23 are utilized. The frequency bands outside the carrier band are also known as information bands.

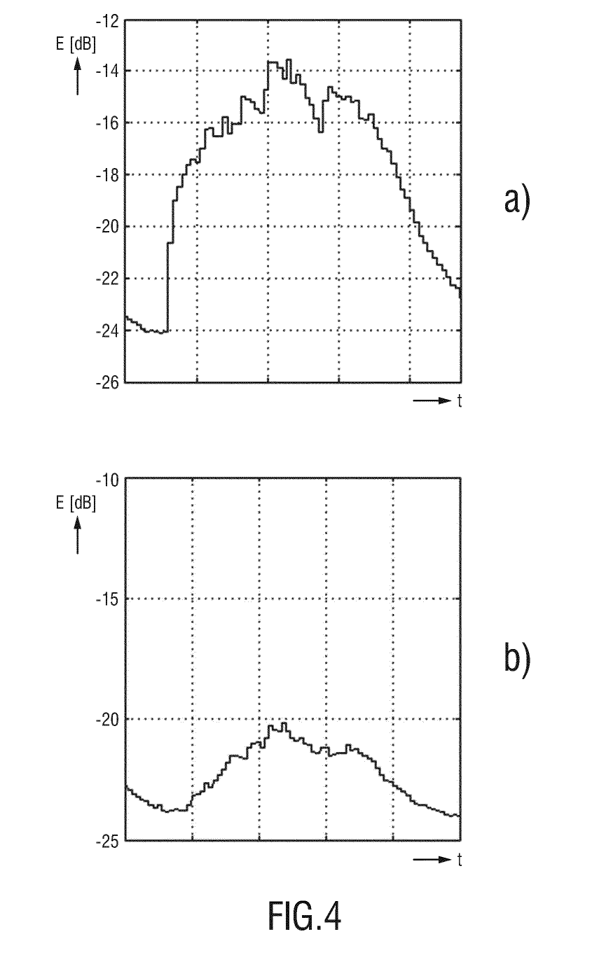

In one embodiment of the invention an average cumulative energy in the information band over time is determined. The corresponding energy of the information band of the ultrasound signal 24 over time are shown in FIGS. 4A, B, wherein the energy shown in FIG. 4A corresponds to a normal unimpaired articulatory movement and the energy shown in FIG. 4B corresponds to an impaired articulatory movement. FIGS. 4A, B show that an impairment result in a reduced average cumulative energy.

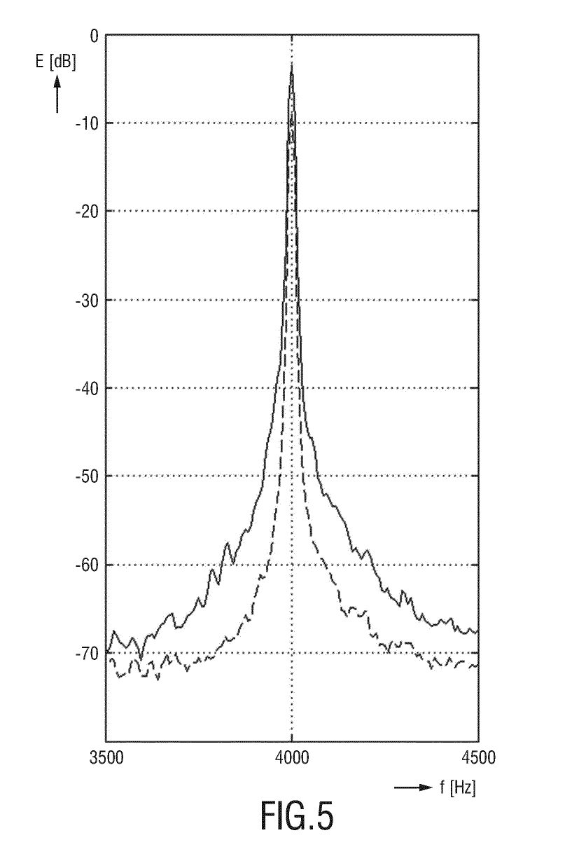

In another embodiment of the invention an average energy spectrum is determined on the basis of all frequency bins. A corresponding average energy spectrum is shown in FIG. 5. The peak at 4 kHz corresponds to the carrier frequency and outside the carrier frequency (4 kHz±50 Hz) the energy of the reflected ultrasound waves or the ultrasound signal 24 is larger for normal articulatory movement (solid line) and the energy of the impaired articulatory movement (dashed line) is reduced. The area below the average energy spectrum outside the carrier can be used or determined as a characteristic feature to distinguish an unimpaired and impaired articulatory movement.

On the basis of these characteristic features determined from the ultrasound signal 24, a robust detection of articulatory degradation can be determined.

Hence, a precise and comparable and reproducible measurement of articulatory movement can be provided.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. A processor or a processing unit is one example of a controller which employs one or more microprocessors that may be programmed using software (e.g., microcode) to perform the required functions. A controller may however be implemented with or without employing a processor, and also may be implemented as a combination of dedicated hardware to perform some functions and a processor (e.g., one or more programmed microprocessors and associated circuitry) to perform other functions.

Computer program code for carrying out the methods of the present invention by execution on the processing unit 26 may be written in any combination of one or more programming languages, including an object oriented programming language such as Java, Smalltalk, C++ or the like and conventional procedural programming languages, such as the “C” programming language or similar programming languages. The program code may execute entirely on the processing unit 26 as a stand-alone software package, e.g. an app, or may be executed partly on the processing unit 26 and partly on a remote server. In the latter scenario, the remote server may be connected to the head-mountable computing device through any type of network, including a local area network (LAN) or a wide area network (WAN), or the connection may be made to an external computer, e.g. through the Internet using an Internet Service Provider.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions to be executed in whole or in part on the processing unit 26, such that the instructions create means for implementing the functions/acts specified in the flowchart and/or block diagram block or blocks. These computer program instructions may also be stored in a computer-readable medium that can direct the cardiopulmonary resuscitation guidance system including the portable computing device to function in a particular manner.

The computer program instructions may, for example, be loaded onto the portable computing device to cause a series of operational steps to be performed on the portable computing device and/or the server, to produce a computer-implemented process such that the instructions which execute on the portable computing device and/or the server provide processes for implementing the functions/acts specified in the flowchart and/or block diagram block or blocks. The computer program product may form part of a patient monitoring system including a portable computing device.

Examples of controller components that may be employed in various embodiments of the present disclosure include, but are not limited to, conventional microprocessors, application specific integrated circuits (ASICs), and field-programmable gate arrays (FPGAs).

In various implementations, a processor or controller may be associated with one or more storage media such as volatile and non-volatile computer memory such as RAM, PROM, EPROM, and EEPROM. The storage media may be encoded with one or more programs that, when executed on one or more processors and/or controllers, perform at the required functions. Various storage media may be fixed within a processor or controller or may be transportable, such that the one or more programs stored thereon can be loaded into a processor or controller.

Any reference signs in the claims should not be construed as limiting the scope.

1. Ultrasound apparatus for determining a medical condition of a subject, comprising:
an ultrasound acquisition unit for receiving ultrasound waves reflected from the subject and for providing an ultrasound signal corresponding to articulatory movements of the subject,
a processing unit coupled to the ultrasound acquisition unit for determining a frequency change of the ultrasound signal over time, and
an evaluation module for evaluating the frequency change of the ultrasound signal and for determining the medical condition of the subject on the basis of the frequency change of the ultrasound signal over time.

2. Ultrasound apparatus as claimed in claim 1, wherein the frequency change of the ultrasound signal corresponds to a movement of the subject or a movement of a portion of the subject.

3. Ultrasound apparatus as claimed in claim 1, wherein the ultrasound acquisition unit is adapted to receive the ultrasound waves contactless.

4. Ultrasound apparatus as claimed in claim 1, wherein the processing unit is adapted to determine an amplitude and/or a velocity of the articulatory movement on the basis of the ultrasound signal and to determine the medical condition on the basis of the amplitude and/or the velocity.

5. Ultrasound apparatus as claimed in claim 1, wherein the processing unit comprises a frequency analysis unit for determining different frequency values of the ultrasound signals, and for determining the medical condition on the basis of the different frequency values.

6. Ultrasound apparatus as claimed in claim 1, wherein the processing unit is further adapted to determine a cumulative energy of the ultrasound signals and to determine the medical condition on the basis of the cumulative energy.

7. Ultrasound apparatus as claimed in claim 6, wherein the processing unit is adapted to determine the cumulative energy of the ultrasound signal as a time dependent cumulative frequency energy.

8. Ultrasound apparatus as claimed in claim 1, wherein the processing unit is adapted to determine an energy spectrum on the basis of the ultrasound signal and to determine the medical condition on the basis of the energy spectrum.

9. Ultrasound apparatus as claimed in claim 8, wherein the processing unit is adapted to determine a variance value of the energy spectrum and to determine the medical condition on the basis of the variance value.

10. Ultrasound apparatus as claimed in claim 1, further comprising a data interface for connecting the evaluation unit to a storage unit and for receiving results of a previous measurement of articulatory movements of the subject, wherein the evaluation unit is adapted to determine the medical condition of the subject on the basis of the frequency change and the results of the previous measurement.

11. Ultrasound apparatus as claimed in claim 1, further comprising a sound detection device for detecting sound received from the subject and for determining an acoustic activity of the subject.

12. Ultrasound apparatus as claimed in claim 1, wherein the evaluation unit comprises an evaluation model for determining the medical condition of the subject on the basis of the frequency change of the ultrasound signal.

13. Method for determining a medical condition of a subject, comprising the steps of:
receiving an ultrasound signal from an ultrasound acquisition unit corresponding to articulatory movements of a subject,
determining a frequency change of the ultrasound signal over time, and
evaluating the ultrasound signal and determining the medical condition of the subject on the basis of the frequency change of the ultrasound signal over time.

14. Ultrasound system for determining a medical condition of a subject, comprising:
an ultrasound apparatus according to claim 1,
an ultrasound transducer including an ultrasound emitter for emitting ultrasound waves, and
a data interface for connecting the evaluation module of the ultrasound apparatus to a storage unit and for receiving results of a previous measurement of articulatory movements of the subject, wherein the evaluation module is adapted to determine the medical condition of the subject on the basis of the frequency change of the ultrasound signal over time and the results of the previous measurement.

15. A computer program product comprising a computer readable medium having computer readable code embodied therein, the computer readable code being configured such that, on execution by a suitable computer or processing unit, the computer or processing unit is caused to perform the method of claim 13.
